(19) United States
(12) Patent Application Publication
(10) Pub. No.: US 2021/0361268 A1
(43) Pub. Date: Nov. 25, 2021

(54) MEDICAL POWER TOOL

(71) Applicant: SHINANO KENSHI KABUSHIKI KAISHA, Nagano (JP)

(72) Inventor: Katsuya OSAWA, Nagano (JP)

(21) Appl. No.: 17/227,510

(22) Filed: Apr. 12, 2021

(30) Foreign Application Priority Data
May 21, 2020 (JP) 2020-088607

(51) Int. Cl. A61B 17/00

(52) U.S. Cl. CPC A61B 17/00; A61B 17/8875; A61B 2017/00398; A61B 2017/0046

(57) ABSTRACT

A medical power tool includes a motor, a first drive transmission part drive-transmitted from the motor, a second drive transmission part drive-transmitted from the first drive transmission part in a non-contact manner, a tool holding part holding a tool drive-transmitted from the second drive transmission part, a tool body having heat resistance, which is assembled so as to cover the first drive transmission part and the second drive transmission part, and the tool detachably mounted to the tool holding part and provided to protrude at a tip side in a longitudinal direction of the tool body.

### CROSS-REFERENCE TO RELATED APPLICATION

This application is based upon and claims the benefit of priority of the prior Japanese Patent Application No. 2020-088607, filed on May 21, 2020, and the entire contents of which are incorporated herein by reference.

### TECHNICAL FIELD

The present invention relates to a medical power tool used after sterilization treatment in medical practice such as surgical operations.

### BACKGROUND ART

In skull reconstructive surgery, orthopedic surgery, and the like in brain surgery, techniques using medical power tools, such as drilling screw holes in a bone by an electric drill or fastening screws by an electric screwdriver are applied for fixing an implant or wire mesh to the bone for the purposes of reducing burdens on patients and doctors and shortening surgery time.

Surgical machinery and equipment used at medical institutions have to be used after sterilization treatment. Sterilization methods include high-pressure steam sterilization (autoclave), ethylene oxide gas sterilization, plasma sterilization, and so on. Characteristics of respective sterilization methods are shown in Table 1 below.

TABLE 1

| | | | | Pressure | Pressure |
|---|---|---|---|---|---|
| | Sterilization | | | (during | (during |
| | time | | Temperature | sterilization) | evacuation) |
| High-pressure | 3-30 minutes | | 115-135° C. | approximately | −0.95M-0.99 Mpa |
| steam | (according to | | is | 0.069-0.222 Mpa | |
| sterilization | sterilization | | mainstream | | |
| (autoclave) | temperature) | | | | |
| EO-gas | 2-4 | hours | 35-60° C. | approximately | Approximately |
| sterilization | | | | 0.090-0.110 Mpa | −0.90 Mpa |
| (EO-gas | | | | | |
| concentration | | | | | |
| 10-30%) | | | | | |
| Plasma | 45-60 | minutes | 45-55° C. | approximately | −0.95M-0.99 Mpa |
| sterilization | | | | 0.100-0.120 Mpa | |

Most of motors and electronic components for driving the motors which are generally distributed do not withstand a high temperature of 135° C. In a case where an autoclave treatment is executed in medical equipment using components not withstanding high temperatures, it is necessary to temporarily remove the components not withstanding the high temperature before treatment. For example, in order to transmit torque of a motor of the electric screwdriver to a bit of the electric screwdriver, a rotating shaft of the motor has to be connected to the bit by using a coupling mechanism. When the coupling mechanism is a method of fixing the shaft by using screws or the like, removing the motor is not realistic, and the fact is that there exists a few electric screwdrivers which conform to the autoclave and can be used plural times.

In consideration of the current state, adoption of new sterilization equipment for gas sterilization or plasma sterilization as low-temperature treatment is selected or a disposable electric screwdriver is selected from the first under acceptance of waste disposal costs at medical institutes (refer to PTL 1: JP-A-2016-5559).

### SUMMARY OF INVENTION

### Technical Problem

When using the method of the above high-pressure steam sterilization (autoclave), to dispose of the medical power tool after using once leads to inefficient use of the tool or a drive source, and also increases medical costs. When using various types of gas sterilization methods, medical institutes have to provide dedicated gas sterilization equipment by capital investment, which may hinder the medical power tool from being widely used.

### Solution to Problem

In response to the above issue, one or more aspects of the present invention are directed to a medical power tool having excellent handleability and capable of reusing many components even after sterilization treatment is executed by using high-pressure steam sterilization (autoclave).

Disclosure relating to embodiments described below includes at least the following configuration. A medical power tool used after sterilization treatment in medical practice includes a motor, a first drive transmission part drive-transmitted from the motor, a second drive transmission part drive-transmitted from the first drive transmission part in a non-contact manner, a tool holding part holding a tool drive-transmitted from the second drive transmission part, a tool body having heat resistance, which is assembled so as to cover the first drive transmission part and the second drive transmission part, and the tool detachably mounted to the tool holding part and provided to protrude at a tip side in a longitudinal direction of the tool body.

According to the above, components not withstanding high temperatures such as the motor and the first drive transmission part can be easily detached before the autoclave treatment and can be reused without damaging components by thermal demagnetization and the like. Moreover, the tool, the tool holding part, the second drive transmission part, and the tool body which are used close to patients can be reused after the autoclave treatment; therefore, medical costs including component costs can be reduced. As the drive transmission is performed from the first drive transmission part to the second drive transmission part in the non-contact manner, the power tool can be easily disassembled before the autoclave treatment, and disposable components can be reduced as few as possible. As many medical institutions are provided with an autoclave sterilization device, the medical power tool according to the present invention can be widely used.

It is preferable that the first drive transmission part and the second drive transmission part are connected by magnet couplings in which annular magnets are concentrically arranged around a motor axis on inner and outer sides in a radial direction.

According to the above, the first drive transmission part and the second drive transmission part are drive-transmitted by using the magnet couplings; therefore, the first drive transmission part and the second drive transmission part can be separated and detached easily, and the power tool can be disassembled easily before the autoclave treatment.

It is preferable that the first drive transmission part includes a cylindrical first yoke member integrally assembled to a motor shaft and the drive-side magnets provided in an annular shape on an inner peripheral surface of the first yoke member, that the second drive transmission part includes a second yoke member integrally connected to the tool holding part and the driven-side magnets provided in an annular shape on an outer peripheral surface of the second yoke member, and that the drive-side magnets and the driven-side magnets are arranged to face each other in the radial direction and magnetically coupled.

When adopting the cylindrical magnet couplings, a transmission torque can be arbitrarily adjusted according to lengths of drive-side and driven side magnets to be coupled. Moreover, as benefits of magnet couplings in the concentric arrangement on inner and outer sides in the radial direction, the tool body can be formed like a slim pencil and the operability can be improved because the transmission torque is adjusted according to lengths of the magnets.

A drive unit including the motor, a power supply unit supplying the power to the motor, and a control substrate and a bearing holder supporting the tool holding part so as to rotate are assembled on both sides in a longitudinal direction of the tool body in a sealed state through sealing materials.

Accordingly, sealability of components housed inside the tool body can be improved.

### Advantageous Effects of Invention

According to the above, it is possible to provide a pencil-type medical power tool having excellent handleability and capable of reusing many components with a few disposable components even after sterilization treatment is executed by using high-pressure steam sterilization (autoclave).

### BRIEF DESCRIPTION OF DRAWINGS

FIG. 1 is a perspective view of an electric screwdriver.

FIG. 2 is a sectional view in an axial direction of the electric screwdriver of FIG. 1.

FIGS. 3A to 3C are sectional views of relevant parts showing mounted/detached states of a driver bit to/from the electric screwdriver of FIG. 2.

FIG. 4 is an exploded perspective view of the electric screwdriver of FIG. 1.

### DESCRIPTION OF EMBODIMENTS

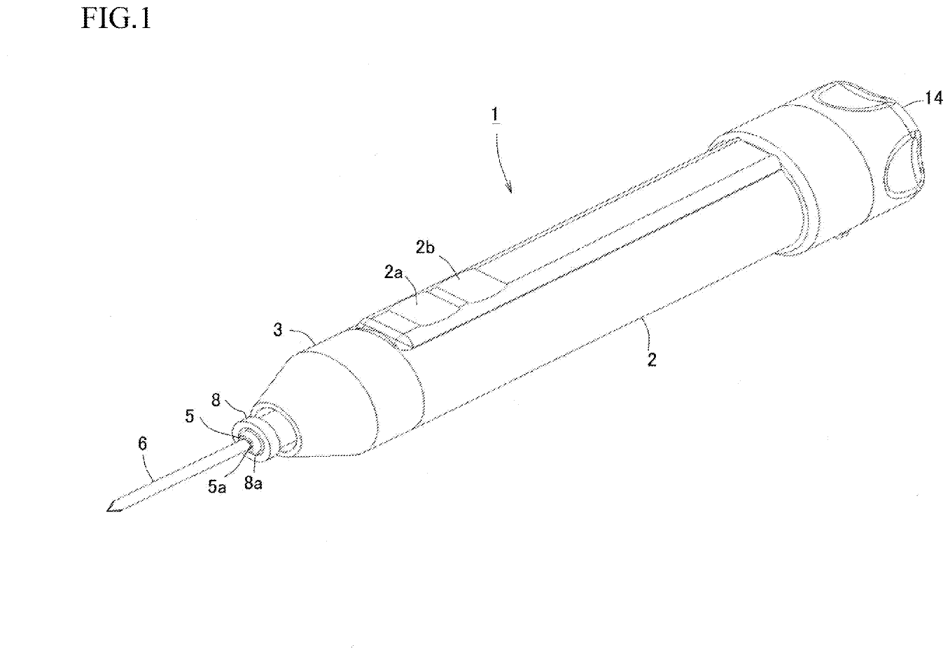

Hereinafter, a medial power tool according to an embodiment of the present invention will be explained with reference to the attached drawings. First, a schematic configuration of an electric screwdriver as an example of a medical power tool used after sterilization treatment by autoclave in medical practice will be explained with reference to FIG. 1 to FIG. 4.

In FIG. 1, an electric screwdriver 1 includes a bearing holder 3 fitted to a cylindrical hole on a tip side of a cylindrical tool body 2. A bit holding part (tool holding part) 5 is supported at the bearing holder 3 so as to rotate. A driver bit (tool) 6 is mounted to the bit holding part 5 while being positioned by a bit chuck 8. An end cap 14 is fitted to a cylindrical hole on a rear-end side of the cylindrical tool body 2.

On an outer peripheral surface of a body part of the tool body 2, operation parts 2a, 2b are provided. The operation part 2a is, for example, a switch for normal rotation and the operation part 2b is, for example, a switch for reverse rotation. These parts may be interchanged with each other. The operation parts 2a, 2b also serve as a substrate cover, and a later-described operation substrate and the like are provided inside the operation parts 2a, 2b.

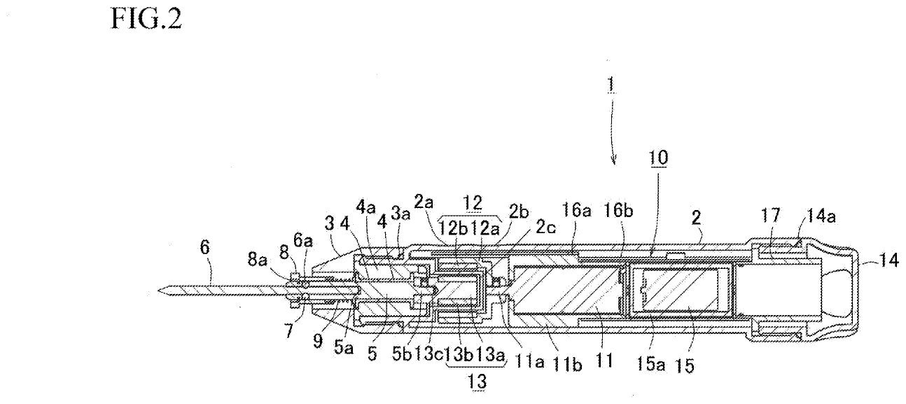

As shown in FIG. 2, the bearing holder 3 is fitted to the tool body 2 through a sealing material 3a. For example, fluororubber or the like having high heat resistance is used for the sealing material 3a. For the tool body 2, resin materials having high heat resistance such as PPSU (polyphenylsulfone resin), PES (polyethersulfone resin), and PEEK (polyetheretherketone) materials so as to withstand an autoclave treatment. A pair of sleeve bearings 4 are assembled inside the bearing holder 3 by being held by a bearing holding member 4a as shown in FIG. 2. Bearings made of heat-resistant resin such as fluororesin or the PEEK material is used as the sleeve bearings 4. The bit holding part (tool holding part) 5 is inserted into the bearing holder 3 and supported by the sleeve bearings 4 so as to rotate.

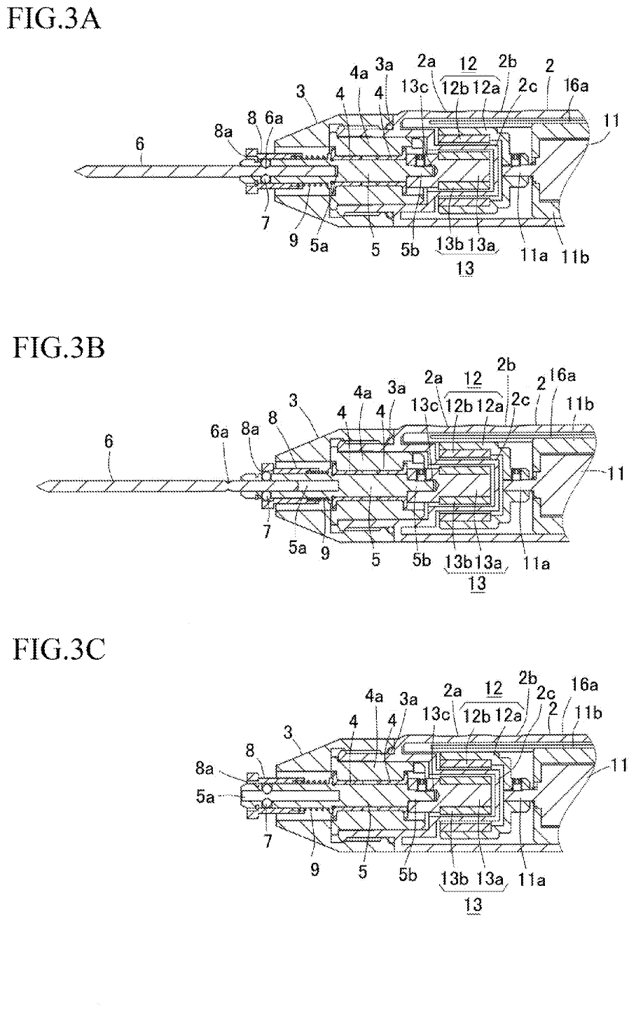

A fitting hole 5a to which the driver bit (tool) 6 is fitted is provided on a tip side of the bit holding part 5 as shown in FIG. 3C. Positioning balls (steel ball) 7 are embedded at a notch part on the tip side of the bit holding part 5. The cylindrical bit chuck 8 is fitted on an outer peripheral side of the positioning balls 7. On an outer periphery on a rear end side of the driver bit 6, a recessed groove 6a to which the positioning balls 7 are fitted is formed. A coil spring 9 is provided between the bit chuck 8 and the sleeve bearings 4. As shown in FIG. 3A, the positioning balls 7 are pushed by an inner peripheral surface of the bit chuck 8 to be fitted to the recessed groove 6a, thereby mounting the drive bit 6 while being positioned in a longitudinal direction with respect to the bit holding part 5 and being stopped from rotating. The bit chuck 8 is biased by the coil spring 9 and held in a state of protruding from a tip of the bearing holder 3. As shown in FIG. 3B, in a case of detaching the driver bit 6, when the bit chuck 8 is pushed back toward the inside of the bearing holder 3 against the biasing of the coil spring 9, the positioning balls 7 are relatively moved to an opening 8a of the bit chuck 8 which is increased in diameter and come out from the recessed groove 6a. Then, only the driver bit 6 can be pulled out from the fitting hole 5a of the bit holding part 5 in a state where the positioning balls 7 are not fitted into the recessed groove 6a even when the driver bit 6 is slid in the longitudinal direction.

When the driver bit 6 is newly mounted, a rear end part of the driver bit 6 is inserted into the fitting hole 5a of the bit holding part 5 in a state of FIG. 3C. Then, the bit chuck 8 is pushed into the inside of the bearing holder 3 once against the biasing of the coil spring 9. While the bit chuck 8 returns to the original position by the biasing of the coil spring 9, the positioning balls 7 are fitted into the recessed groove 6a as shown in FIG. 3A, thereby mounting the driver bit 6 while being positioned in the longitudinal direction.

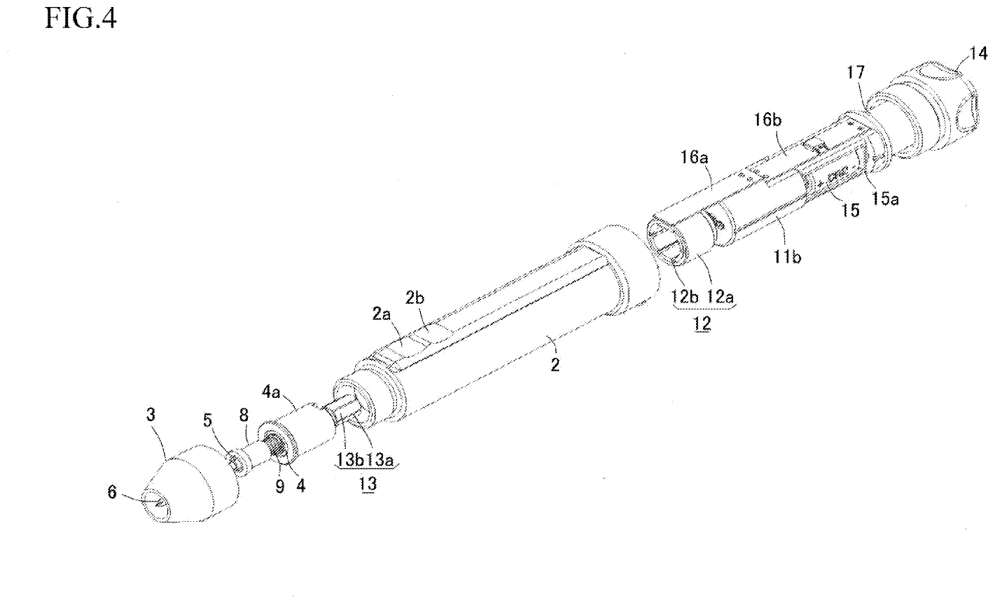

As shown in FIG. 2, a drive unit 10 is inserted in a rear-end side cylindrical hole of the tool body 2. The drive unit 10 has the following configuration. A motor 11 is housed in the cylindrical hole of the tool body 2 through a motor case 11b. A brushless motor is preferably used for the motor 11. In the motor 11, DC power is supplied from a battery (a power supply unit) 15 arranged in series in the longitudinal direction of the tool body 2. The battery 15 is housed in a battery case 15a, and an operation substrate 16a and a control substrate 16b are provided on an outer peripheral side of the motor case 11b and the battery case 15a. The operation substrate 16a is provided so as to correspond to the operation parts 2a, 2b provided on the outer peripheral surface of the body part of the tool body 2, which is fixed to the motor case 11b by screws (see FIG. 4). The control substrate 16b is provided so as to correspond to the motor 11 and the battery 15, which is fixed to the motor case 11b and a later-described retaining member 17 by screws respectively (see FIG. 4). The motor 11 is driven to rotate in a prescribed direction (a normal rotation direction or a reverse rotation direction) only while a user presses any of the operation parts 2a, 2b. The retaining member 17 for retaining the drive unit 10 is fitted to the rear end side of the tool body 2 adjacent to the battery 15. The end cap 14 is filled to the retaining member 17 through a sealing material 14a, thereby assembling the drive unit 10 inside the tool body 2 in a sealed state. As the sealing material 14a, fluororubber or the like having heat resistance is used.

A first drive transmission part 12 is assembled to a motor shaft 11a of the motor 11. Specifically, a first yoke member 12a having a bottomed cylindrical shape is integrally assembled to the motor shaft 11a. First magnets 12b (drive-side magnets) provided in an annular shape are integrally assembled to an inner peripheral surface of the first yoke member 12a (magnetic material). A rear end side of the bit holding part 5 is extended from the bearing holder 3 to the tool body 2 side, which is assembled so as to be fitted to a second drive transmission part 13 by a screw. Specifically, a screw hole 13c is provided at a tip part of a second yoke member 13a, and a screw part 5b provided at a rear end part of the bit holding part 5 is screwed to the screw hole 13c to be assembled.

The second drive transmission part 13 is arranged to face the first drive transmission part 12, and drive transmission is performed in a non-contact manner. The second drive transmission part 13 includes the bar-shaped second yoke member 13a (magnetic material) which is screw-fitted with the screw part 5b of the bit holding part 5 and annular-shaped second magnets 13b (driven-side magnets) assembled to an outer periphery of the second yoke member 13a. The second magnets 13b and the first magnets 12b are concentrically arranged around a motor axis. In the first magnets 12b and the second magnets 13b, N-poles and S-poles are alternately magnetized in a circumferential direction. The second magnets 13b are arranged on an inner side in a radial direction and the first magnets 12b arranged on an outer side in the radial direction, and magnet couplings in which different magnetic poles are arranged to face each other and magnetically coupled are formed. Permanent magnets such as metal magnets (alnico magnet, rare earth magnets (neodymium magnet, samarium-cobalt magnet, and the like) and bonded magnets are used for the first magnets 12b and the second magnets 13b. The first magnets 12b and the second magnets 13b are arranged to face each other through a partition wall 2c provided inside the cylindrical hole of the tool body 2. Accordingly, the tool body 2 can be shaped like a pencil, which improves operability.

As shown in FIG. 2, when the operation part 2a or the operation part 2b of the tool body 2 is pressed, the motor 11 is activated and the first drive transmission part 12 (the first yoke member 12a and the first magnets 12b) assembled to the motor shaft 11a rotates in a prescribed direction. The second magnets 13b magnet-coupled to the first magnets 12b through the partition wall 2a are driven to rotate, then, the bit holding part 5 integrally connected to the second yoke member 13b and the driver bit 6 held by the bit holding part 5 integrally rotate.

According to the above, as shown in FIG. 4, components not withstanding high temperatures such as the drive unit 10 (the battery 15, the motor 11, the operation substrate 16a, the control substrate 16b) and the first drive transmission part 12 can be easily detached before the autoclave treatment and reused by removing the end cap 14 from the tool body 2, and the autoclave treatment can be performed without damaging other components having heat resistance by thermal demagnetization due to high temperature. Specifically, the driver bit 6 (tool), the bearing holder 3, the bit holding part 5, the second drive transmission part 13, the tool body 2 and the like which are used close to humans receive the autoclave treatment. As a result, much components (the driver bit 6, the bearing holder 3, the tool holding part 5, the second drive transmission part 13, the tool body 2, the first drive transmission part 12, the drive unit 10 and the like) can be reused except consumable parts; therefore, medical costs can be reduced. Moreover, drive transmission is performed from the first drive transmission part 12 to the second drive transmission part 13 in the non-contact manner; therefore, the electric screwdriver 1 can be easily disassembled before the autoclave treatment, and disposable components can be reduced as few as possible.

Additionally, drive transmission is performed between the first drive transmission part 12 and the second drive transmission part 13 by using magnet couplings; therefore, the first drive transmission unit 12 can be separated from the second drive transmission unit 13 without contact and detached from the tool body 2 with the drive unit 10 only by removing the end cap 14 from the tool body 2, and the second drive transmission part 13 can be taken out with the tool holding part 5 only by removing the bearing holder 3 from the tool body 2. Accordingly, the electric screwdriver 1 can be easily disassembled before the autoclave treatment.

When cylindrical magnet couplings are adopted, a transmission torque can be arbitrarily adjusted according to lengths of couplings. As benefits of the couplings of the same shape, the tool body 2 can be formed like a slim pencil and the operability can be improved because the transmission torque is adjusted according to lengths.

The electric screwdriver 1 is cited above as an example of the power tool, and the tool to be mounted to the bit holding part 5 may be changed to be used from the driver bit 6 to other tools such as a drill bit.

What is claimed is:

1. A medical power tool used after sterilization treatment in medical practice, comprising:
a motor;
a first drive transmission part drive-transmitted from the motor;
a second drive transmission part drive-transmitted from the first drive transmission part in a non-contact manner;
a tool holding part holding a tool drive-transmitted from the second drive transmission part;
a tool body having heat resistance, which is assembled so as to cover the first drive transmission part and the second drive transmission part; and
the tool detachably mounted to the tool holding part and provided to protrude at a tip side in a longitudinal direction of the tool body.

2. The medical power tool according to claim 1,
wherein the first drive transmission part and the second drive transmission part are connected by magnet couplings in which annular magnets are concentrically arranged around a motor axis on inner and outer sides in a radial direction.

3. The medical power tool according to claim 1,
wherein the first drive transmission part includes a cylindrical first yoke member integrally assembled to a motor shaft and the drive-side magnets provided in an annular shape on an inner peripheral surface of the first yoke member,
the second drive transmission part includes a second yoke member integrally connected to the tool holding part and the driven-side magnets provided in an annular shape on an outer peripheral surface of the second yoke member, and
the drive-side magnets and the driven-side magnets are arranged to face each other in the radial direction and magnetically coupled.

4. The medical power tool according to claim 1,
wherein a drive unit including the motor, a power supply unit supplying the power to the motor, and a control substrate and a bearing holder supporting the tool holding part so as to rotate are assembled on both sides in a longitudinal direction of the tool body in a sealed state through sealing materials.
